(19) United States
(12) Patent Application Publication
(10) Pub. No.: US 2023/0346595 A1
(43) Pub. Date: Nov. 2, 2023

(54) DEVICES AND METHODS FOR IMPROVING IDENTIFICATION AND VISUALIZATION OF MEMBRANES

(71) Applicant: Alcon Inc., Fribourg (CH)

(72) Inventor: Paul R. Hallen, Colleyville, TX (US)

(21) Appl. No.: 18/303,133

(22) Filed: Apr. 19, 2023

Related U.S. Application Data
(60) Provisional application No. 63/363,740, filed on Apr. 28, 2022.

(51) Int. Cl. A61F 9/00; A61F 9/007; A61K 49/00

(52) U.S. Cl. CPC A61F 9/0008; A61F 9/007; A61K 49/006

(57) ABSTRACT

Certain embodiments herein relate to a method for staining membranes of an eye. In certain embodiments, the method includes mixing a first dye and a second dye to obtain a combination dye, wherein the first dye comprises a first color for staining a first membrane in the eye and the second dye comprises a second color for staining a second membrane in the eye. The method further includes delivering the combination dye to the eye to stain the first membrane with the first color and a second membrane with the second color.

### BACKGROUND

During certain ophthalmic surgical procedures a surgeon may be required to manipulate (e.g. remove, cut, peel, etc.) certain tissues, such as an internal limiting membrane (ILM) and/or an epiretinal membrane (ERM), using forceps, scissors, etc., for treating different macular surface diseases. The ILM is the outer membrane of the retina, interfacing with the vitreous humor (vitreous). The ERM is a pathological membrane that may form over the retina as a result of a disease or disorder. The ERM can also form over the macula causing further complications. Identification of the ILM and ERM is a difficult aspect of these ophthalmic surgical procedures. Difficulties in identification may result from difficulties in determining depth in the patient's eye and/or certain properties of the membranes (e.g. colorless, thin, etc.) making the membranes difficult to distinguish from the retina.

### BRIEF SUMMARY

The present disclosure relates generally to methods and devices for improving identification and visualization of membranes in a patient's eye during ophthalmic surgical procedures.

Certain embodiments provide a method for utilizing a combination dye for staining membranes in a patient's eye. The method includes mixing a first dye and a second dye to obtain a combination dye, wherein the first dye comprises a first color for staining a first membrane in the eye and the second dye comprises a second color for staining a second membrane in the eye. The method further includes delivering the combination dye to the eye to stain the first membrane with the first color and the second membrane with the second color.

Certain embodiments provide a method for utilizing a number of dyes for staining membranes in a patient's eye. The method includes delivering, using an injection device, the first dye to stain a first membrane in the eye with a first color. The method further includes delivering, using the injection device, the second dye to stain a second membrane in the eye with a second color.

The following description and the related drawings set forth in detail certain illustrative features of one or more embodiments.

### BRIEF DESCRIPTION OF THE DRAWINGS

The drawings described herein are for illustrative purposes only, are schematic in nature, and are intended to be exemplary rather than to limit the scope of the disclosure.

FIG. 1A shows a cross-sectional view of a patient's eye.

FIG. 1B shows an expanded or zoomed in view of the different layers of the retina at the optic nerve, where signals travel from the retina to the brain.

FIG. 2A shows a flowchart depicting an example method for delivering a combination dye, including a first dye of a first color and a second dye of a second color, to a patient's eye to stain a first membrane with a first color and second membrane with a second color, in accordance with certain embodiments.

FIG. 2B shows a flowchart of an example method for delivering, using an injection device, two dyes comprising different colors sequentially to stain a first and a second membrane in a patient's eye, in accordance with certain embodiments.

FIG. 3A shows a diagram of an example injection device including a source one and a source two connected to a three-way valve with an output infusion line for delivery of one or more dyes into a patient's eye, in accordance with certain embodiments.

FIG. 3B shows a diagram of another example injection device including a first chamber, a second chamber, a plunger for the first chamber, a plunger for the second chamber and a hub for delivery of one or more dyes to a patient's eye, in accordance with certain embodiments.

FIG. 3C shows a diagram of another example injection device including a chamber, a plunger, and a hub for delivery of one or more dyes to a patient's eye, in accordance with certain embodiments.

The above summary is not intended to represent every possible embodiment or every aspect of the subject disclosure. Rather, the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods.

Certain embodiments herein describe systems and methods for utilizing one or more dyes to improve identification and visualization of both the ILM and ERM, e.g., during the same procedure. For example, certain embodiments herein describe systems and methods for utilizing one or more dyes to stain the ILM and ERM with different colors, thereby distinguishing the two for better identification and visualization. Certain embodiments herein further describe an injection device to deliver one or more dyes to the patient's eye to stain the ILM and the ERM.

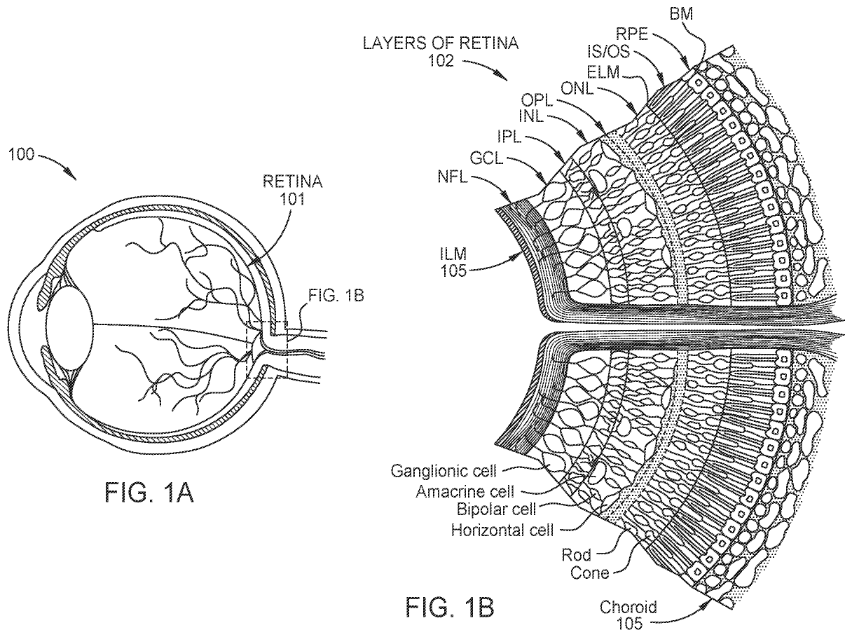

FIG. 1A is a diagram of a cross-sectional view of a patient's eye 100 while FIG. 1B illustrates an expanded or zoomed in view of the different layers of the retina 102 at the optic nerve where signals travel from the retina to the brain. Layers of the retina 102 are shown from the innermost layer, ILM 105, to the outermost layer, choroid 120. The ILM 105 is the first layer of the retina 101, which is also in contact with the vitreous humor. The ERM, though not shown in FIG. 1, is a pathological membrane that may form on top of the ILM 105 and may cause distortion of the retina.

ILM 105 is a thin, colorless, barely visible membrane on the surface of the retina. The ILM's function in adults is unknown but it is known to contribute to a variety of vitreoretinal diseases such as macular hole (MH), macular pucker (MP) and other ophthalmic disorders and diseases. Removing the ILM 105 can improve vision and treat eye disorders, for example, by encouraging MH closure and improving or eliminating MP. Given the thin and colorless properties of the ILM 105, the ILM 105 can be difficult to visualize in ophthalmic surgeries such as an ILM removal procedure.

The ERM is another membrane that may need to be removed to improve a patient's vision. An ERM may form on the surface of the ILM 105 and commonly causes no symptoms, though over time can lead to vision impairment. An ERM may also be known as MP, where the growth of the membrane affects the macula, the central portion of the retina responsible for a patient's vision, causing the macula to contract or wrinkle. In some cases, without surgery to remove the MP, MP can progress causing distortion of the retina and limited vision. The ERM may also be difficult to visualize in surgical procedures.

Whether the goal of the surgical procedure is removal of the ERM, ILM 105 or both membranes, current practices of identifying the membranes and distinguishing them from one another may be ineffective. The embodiments herein provide systems, methods and injection solutions to allow for better identification and visualization of the ILM 105 and ERM, while also distinguishing between the two by staining the ERM and ILM 105 with dyes of different colors. Improved identification and ability to distinguish the ERM and ILM 105 in the patient's eye may improve accuracy and efficiency of ILM 105 and ERM removal.

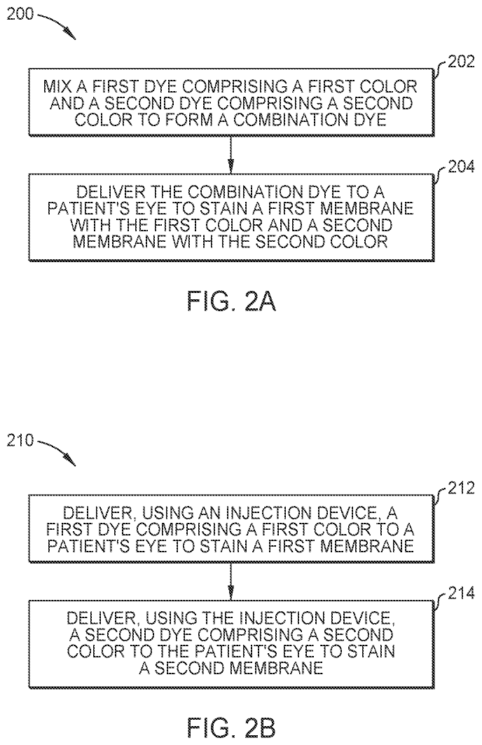

FIG. 2A is a flowchart depicting operations 200 for delivering a combination dye, including a first dye of a first color and a second dye of a second color, to a patient's eye to stain a first membrane with a first color and second membrane with a second color, in accordance with certain embodiments.

At 202, operations 200 begin with mixing a first dye comprising a first color and a second dye comprising a second color to form a combination dye. In some embodiments, the dyes may be mixed in the injection device prior to delivering the combination dye to the patient's eye. In certain other embodiments, the dye may be mixed in a separate device or container and then transferred to the injection device prior to injection into the patient's eye. In certain embodiments, the first dye may be an indo-cyanine green (ICG) dye and the second dye may be a Brilliant Blue G (BBG) dye. In certain embodiments, other dyes with different colors may be used. In some embodiments, the amount of each of the dyes may be 0.5 cubic centimeters (cc) to 5 cc of ICG and BBG, respectively.

At 204, operations 200 continue with delivering the combination dye to a patient's eye to stain a first membrane with the first color and a second membrane with the second color. In certain embodiments, the first color may be green from the ICG dye and the first membrane may be the ILM, while the second color may be blue from the BBG dye and the second membrane may be ERM. In certain other embodiments, the first color may be blue from the BBG dye and the first membrane may be the ILM, while the second color may be green from the ICG dye and the second membrane may be ERM. In certain embodiments, delivering the combination dye may be performed using a single injection device. When injected as a single composition, the combination dye stains the ILM and the ERM with different colors. Note that step 204 may be performed using a syringe or injection device that may be operated manually or by a surgical console, similar to the injection devices described herein.

FIG. 2B is a flowchart of operations 210 for delivering, using an injection device, two dyes comprising different colors simultaneously or sequentially to stain a first and a second membrane in a patient's eye, in accordance with certain embodiments. In certain embodiments, the first dye may be ICG dye and the second dye may be BBG dye. In certain embodiments, other dyes with different colors may be used. In some embodiments, the amount of each of the dyes may be 0.5 cc to 5 cc of ICG and BBG, respectively.

At 212, operations 210 begin with delivering, using an injection device, a first dye comprising a first color to a patient's eye to stain a first membrane.

At 214, operations 210 continue with delivering, using the injection device, a second dye comprising a second color to the patient's eye to stain a second membrane.

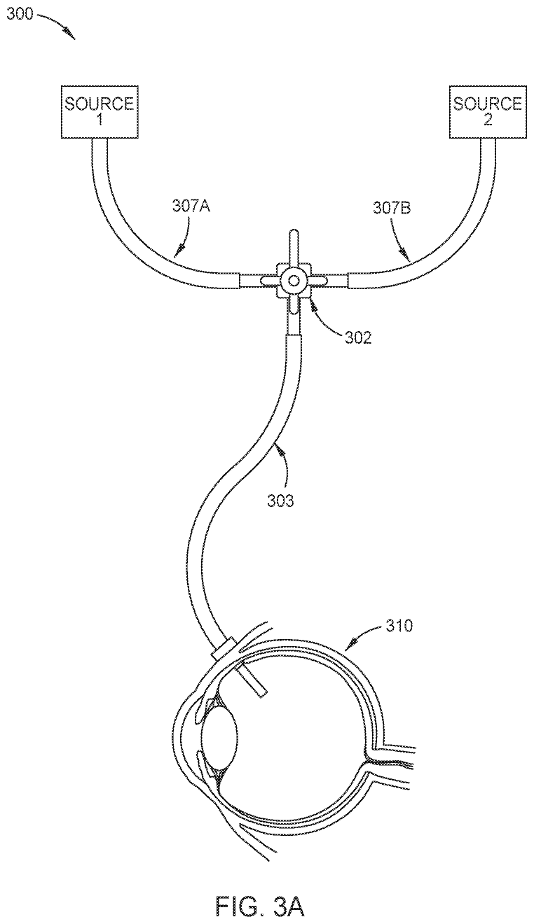

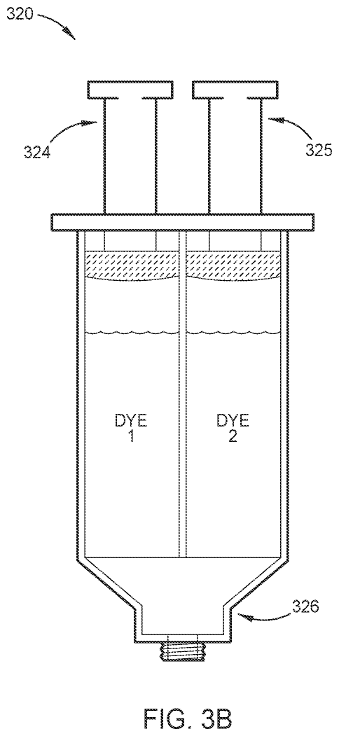

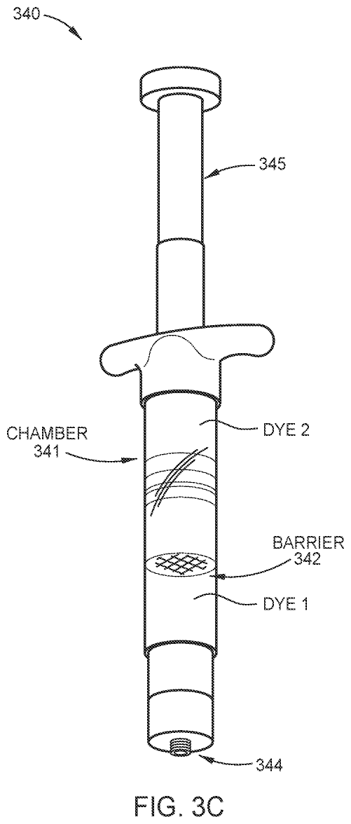

One of a variety of injection devices may be used for simultaneously or sequentially injecting a first and a second dye to stain a first and a second membrane, as described above. Examples of injection devices that may be used in conjunction with operations 210 are illustrated in FIGS. 3A-3C. Each of these exemplary injection devices allows for the injection of the first and second dyes, simultaneously or sequentially, into the patient's eye, such as, during the same ophthalmic surgical procedure and, for example, without removing or having to remove the injection device from the eye in order to inject both dyes.

FIG. 3A is an exemplary injection device 300, including source one and source two connected to a valve 302 with an output infusion line 303 for delivery of one or more dyes into the patient's eye 310. In some embodiments, the valve 302 is a three-way valve as shown in FIG. 3A. In some embodiments, the sources may be two compartments, e.g., syringes, where the compartment in source one holds a dye of a first color and the compartment in source two holds a dye of a second color. The valve 302 comprises two input lines 307A and 307B, where 307A is attached to source one for receiving a first dye and 307B is attached to source two for receiving a second dye. The valve 302 allows for one or both dyes to travel down the output infusion line 303 and into the patient's eye 310.

In particular, the valve 302 allows for the injection of the first and second dyes sequentially into the patient's eye 310 during the same procedure, e.g., without removing or having to remove the output infusion line 303 from the eye in order to inject both dyes. The valve 302 is shown in FIG. 3A in an off position, where neither source one nor source two is providing dye into the patient's eye 310. The valve 302 may be moved from the off position to a position which allows either the dye from source one or the dye from source two to travel through the output infusion line 303 into the patient's eye 310. Note that in certain embodiments, the valve 302 may operate in a manner that allows for the dye from source one and the dye from source two to be injected into the output infusion line 303 and then to the patient's eye 310 simultaneously.

In some embodiments, the valve 302 in the injection device 300 may be manual, where the surgeon or medical professional manually monitors the valve 302 to control when the dye from source one and source two is delivered to the patient's eye 310. In certain other embodiments, the injection device 300 may be connected to and controlled by a surgical console, such that input from, e.g., a surgeon, may cause the console to automatically rotate the valve 302 from the off position and deliver dye from source one or source two into the patient's eye 310.

In some embodiments, the surgical console may be configured with components to allow for automatically controlling the operations of injection device 300. For example, the surgical console may include an actuator that is able to rotate the valve 302 in response to user input. In certain embodiments, the surgical console may include one or more pneumatic devices that use compressed gas, such as nitrogen, to power the injection device. For example, one pneumatic device may cause a first dye to exit out of source one into input line 307A and another pneumatic device may cause a second dye to exit out of source two into input line 307B. In another example, the same pneumatic device may be configured to cause the first dye and the second dye to exit out of source one and source two, respectively. In yet another example, injection device 300 may be operated by adjusting pressure in the source one and source two differently (e.g., increasing pressure in source one and decreasing it in source two). For example, pressure in source one may be increased while pressure in source two may be decreased causing valve 302 to deliver the dye in source one to the eye.

The user input from, e.g., the surgeon, may be a provided through a foot switch or a user interface of the surgical console to, for example, rotate the valve 302 and/or cause the first dye and the second dye to be injected into input lines 307A-307B and then into output infusion line 303.

In some embodiments, the dye in source one may be used to stain the ILM and may comprise ICG. The dye in source two may be used to stain the ERM and may be BBG. In some embodiments, the amount of dye in source one and source two may be 0.5 cc to 5 cc of ICG and BBG, respectively.

FIG. 3B in an exemplary injection device 320 including a first chamber comprising dye 1, a second chamber comprising dye 2, a plunger for the first chamber 324, a plunger for the second chamber 325, and a hub 326 for delivery of one or more dyes to a patient's eye. The tip of hub 326 may be connected to an infusion line which may be inserted into a patient's eye 310 for delivery of dye 1 and dye 2. Note that although injection device 320 is shown as having a single hub 326, in certain other embodiments, two hubs may be coupled to the two chambers and provide the dyes to two separate infusion lines for delivery into the patient's eye 310.

The injection device 320, which operates like a syringe, may be used manually to inject dye 1 and dye 2 into the patient's eye 310. For example, the surgeon depresses the left or right plunger of the syringe to deliver dye 1 or dye 2 to the patient's eye 310. In certain embodiments, the surgeon may depress one plunger at a time, such as to inject dye 1 and dye 2 sequentially. In certain other embodiments, the surgeon may depress both plungers at the same time, such as to inject dye 1 and dye 2 simultaneously during the same procedure.

In some embodiments, the injection device 320 may be coupled to a surgical console where user input may be provided through a foot switch, a user interface display, or a verbal command, for example, for operating injection device 320. When the surgical console receives user input, a device (e.g., pneumatic device) within or coupled to the surgical console may depress one or both plungers 324 and 325 (e.g., simultaneously or sequentially) to deliver dye 1 and/or dye 2 to the patient's eye 310. In embodiments where the a pneumatic device is used for operating injection device 320, such as discussed previously in relation to FIG. 3A, the pneumatic device is configured to trigger pressured gas to depress plunger 324 and/or plunger 325 depending on the user input received. In some embodiments, the user input may correspond to delivering either dye 1 or dye 2, or both, to the patient's eye 310. Note that the use of a pneumatic mechanism is merely provided as an example and that one of a variety of other mechanisms may be used in certain other embodiments for causing injection device 320 to deliver dye 1 and/or dye 2.

In some embodiments, dye 1 may be used to stain the ILM and may comprise ICG. Dye 2 may be used to stain the ERM and may be BBG. In some embodiments, the amount of dye 1 and dye 2 may be 0.5 cc to 5 cc of ICG and BBG, respectively.

FIG. 3C is an exemplary injection device 340 including a chamber 341, hub 344, and plunger 345 for delivery of one or more dyes to a patient's eye. In some embodiments, dye 1 and dye 2 may be present in the chamber 341 in layers separated by a barrier 342. The tip of hub 344 may be connected to an infusion line which may be inserted into the patient's eye 310 for delivery of one or more dyes.

In some embodiments, chamber 341 holds dye 1 and dye 2, which as shown in FIG. 3, are separated by a horizontal barrier 342. The barrier 342 may be made of foil or other material that may be punctured following dye 1 being fully delivered into the patient's eye 310. Once dye 1 is removed from injection device 340, the barrier 342 may be punctured, for example, due to pressure being exerted by dye 2 as the plunger is being depressed causing barrier 342 to be pressed against the tip of hub 344. The punctured barrier 342 allows dye 2 to enter the patient's eye 310 following the delivery of dye 1.

As described in FIG. 3B, dye 1 may be used to stain the ILM and may comprise ICG. Dye 2 may be used to stain the ERM and may comprise BBG. In some embodiments, the amount of dye 1 and dye 2 may be 0.5 cc to 5 cc of ICG or BBG.

In some embodiments, injection device 340 may be used manually where the surgeon or medical professional depresses the plunger 345 to deliver dye 1 and dye 2 sequentially.

In some embodiments, the injection device 340 may be coupled to a surgical console where user input may be provided through a foot switch, a user interface display, or a verbal command, for example, for operating injection device 340. When the surgical console receives user input, a device (e.g., pneumatic device) within or coupled to the surgical console may depress plunger 345 to deliver dye 1 and/or dye 2 to the patient's eye 310. In embodiments where a pneumatic device is used for operating injection device 340, such as discussed previously in relation to FIG. 3A, the pneumatic device is configured to trigger pressured gas to depress plunger 345 depending on the user input received. In some embodiments, the user input may correspond to delivering either dye 1 or dye 2, or both, to the patient's eye 310. Note that the use of a pneumatic mechanism is merely provided as an example and that one of a variety of other mechanisms may be used in certain other embodiments for causing injection device 340 to deliver dye 1 and/or dye 2.

What is claimed is:

1. A method for staining membranes of an eye, comprising:
mixing a first dye and a second dye to obtain a combination dye, wherein:
the first dye comprises a first color for staining a first membrane in the eye;
the second dye comprises a second color for staining a second membrane in the eye; and
delivering the combination dye to the eye to stain the first membrane with the first color and the second membrane with the second color.

2. The method of claim 1, wherein the first dye is an Indo-cyanine Green (ICG) dye and the second dye is a Brilliant Blue G (BBG) dye.

3. The method of claim 1, wherein the first dye stains an internal limiting membrane (ILM) and the second dye stains an epiretinal membrane (ERM).

4. A method for staining membranes of an eye, comprising:
delivering, using an injection device, a first dye to stain a first membrane in the eye with a first color; and
delivering, using the injection device, a second dye to stain a second membrane in the eye with a second color.

5. The method of claim 4, wherein the first dye is an Indo-cyanine Green (ICG) dye and the second dye is a Brilliant Blue G (BBG) dye.

6. The method of claim 4, wherein the first dye stains an internal limiting membrane (ILM) and the second dye stains an epiretinal membrane (ERM).

7. The method of claim 4, wherein the injection device comprises:
a first source comprising the first dye;
a second source comprising the second dye;
a three-way valve, wherein the first source and the second source are connected to the three-way valve by two separate input lines; and
an output infusion line connecting an output of the three-way valve to the eye.

8. The method of claim 7, wherein the three-way valve is configured to sequentially or simultaneously deliver the first dye from the first source and the second dye from the second source to the eye through the output infusion line.

9. The method of claim 4, wherein the injection device is a syringe comprising:
a first chamber comprising the first dye;
a second chamber comprising the second dye;
a first plunger configured to deliver the first dye in the first chamber through a hub to the eye;
a second plunger configured to deliver the second dye in the second chamber through the hub to the eye; and
the hub for delivery of the first and the second dyes to the eye.

10. The method of claim 9, wherein the first and second dyes are configured to be delivered sequentially.

11. The method of claim 9, wherein the first and second dyes are configured to be delivered simultaneously.

12. The method of claim 4, wherein the injection device is a syringe comprising:
a chamber comprising the first dye and the second dye;
a plunger;
a barrier to separate the first dye from the second dye in the chamber; and
a hub for delivering the first dye and the second dye to the eye.

13. The method of claim 12, wherein depressing the plunger to fully deliver the first dye causes the barrier to puncture thereby allowing the second dye to be delivered to the eye through the hub.
